(19) United States
(12) Patent Application Publication
(10) Pub. No.: US 2007/0129800 A1
(43) Pub. Date: Jun. 7, 2007

(54) Hydrolic accommodating intraocular lens

(75) Inventor: J. Stuart Cumming, Laguna Beach, CA (US)

Correspondence Address: ORRICK, HERRINGTON & SUTCLIFFE, LLP;IP PROSECUTION DEPARTMENT, 4 PARK PLAZA, SUITE 1600, IRVINE, CA, 92614-2558, US

(73) Assignee: C&C VISION INTERNATIONAL LIMITED

(21) Appl. No.: 11/297,232

(22) Filed: Dec. 7, 2005

(51) Int. Cl. A61F 2/16

(52) U.S. Cl. 623/6.13; 623/6.37; 623/6.44

(57) ABSTRACT

An accommodating intraocular lens comprising an optic made from solid silicone and liquid silicone. The optic has a central anterior area or membrane that can vary in radius and thus charge power.

### BACKGROUND

Intraocular lenses have for many years had a design of a single optic with loops attached to the optic to center the lens and fixate it in the empty capsular bag of the human lens. In the mid '80s plate lenses were introduced, which comprised a silicone lens, 10.5 mm in length, with a 6 mm optic. These lenses could be folded but did not fixate well in the capsular bag, but resided in pockets between the anterior and posterior capsules. The first foldable lenses were all made of silicone. In the mid 1990s an acrylic material was introduced as the optic of lenses. The acrylic lens comprised a biconvex optic with a straight edge into which were inserted loops to center the lens in the eye and fixate it within the capsular bag.

Recently accommodating intraocular lenses have been introduced to the market, which generally are modified plate haptic lenses and, like the silicone plate haptic lenses, have no clear demarcation between the junction of the plate with the optic's posterior surface. A plate haptic lens may be referred to as an intraocular lens having two or more plate haptics joined to the optic.

Flexible acrylic material has gained significant popularity among ophthalmic surgeons. In 2003 more than 50% of the intraocular lenses implanted had acrylic optics. Hydrogel lenses have also been introduced. Both the acrylic and hydrogel materials are incapable of multiple flexions without fracturing.

The advent of an accommodating lens which functions by moving along the axis of the eye by repeated flexions somewhat limited the materials from which the lens could be made. Silicone is the ideal material, since it is flexible and can be bent probably several million times without showing any damage. Additionally a groove or hinge can be placed across the plate adjacent to the optic as part of the lens design to facilitate movement of the optic relative to the outer ends of the haptics. On the other hand, acrylic material fractures if it is repeatedly flexed.

An example accommodating lens is a type as disclosed in U.S. Pat. No. 6,387,126 and others in the name of J. Stuart Cumming.

### SUMMARY OF THE INVENTION

According to a preferred embodiment of this invention, an accommodating lens comprises a lens with a flexible solid and interior liquid optic, preferably with two or more extended portions from the solid optic which may be plate haptics capable of multiple flexions without breaking, preferably along with fixation and centration features at their distal ends. There may be a hinge or groove across the extended portions adjacent to the optic to facilitate the anterior and posterior movement of the optic relative to the outer ends of the extended portions. On the other hand, the optic may be rigidly attached to the haptics. Also, haptics can be omitted.

According to the present invention the optic is of a foldable, flexible silicone, acrylic or hydrogel material with an interior of liquid silicone, and the haptics are of a foldable material that will withstand multiple foldings without damage, e.g., silicone. Preferably, the end of the plate haptics have T-shaped fixation devices and the haptics are hinged to the optic.

The lens of the present invention is made of solid silicon with liquid silicone both of which have the same refractive index, and have a specific gravity the same as or very similar to that of the aqueous solution of the natural eye. The power of the lens, before implantation into the eye, can be changed by (1) changing the radius of a posterior portion of the optic, and/or (2) by changing the volume of the liquid silicone in the lens optic during the manufacturing process. During accommodation with contraction of the ciliary muscle and an increase in the vitreous cavity pressure the posterior surface of the solid silicone portion of the lens is pushed forward. This causes bulging of a thin anterior membrane thereby increasing its curvature, thus decreasing the radius of the anterior surface of the lens, for near vision. Also, the increase in vitreous cavity pressure can tilt the lens to further facilitate accommodation.

Accordingly, features of the present invention are to provide an improved form of accommodating lens formed from solid and liquid silicone.

### BRIEF DESCRIPTION OF THE DRAWINGS

FIG. 1 is a side cross sectional view of the preferred embodiment of the lens of the present invention.

FIG. 2 is a plan view from the posterior side of the lens.

FIG. 3 is a plan view from the anterior side of the lens.

FIG. 4 is a cross-sectional view of the lens like FIG. 1 but showing bulging or increased curvature of an anterior portion of the lens.

### DESCRIPTION OF THE PREFERRED EMBODIMENT

Turning now to the drawings, a preferred embodiment is shown in detail, comprising an intraocular lens with an optic 10 and haptics 16. The optic 10 is formed of two components, namely, a flexible solid portion 12 (12a-12d) preferably made of silicone, acrylic or hyrdrogel, and an interior liquid silicone portion 14. The portions 12a and 12b are sufficiently solid to prevent defomation of the optic 10 upon implantation into the fibrosed capsular bag of the eye. The flexible extending portions 16 may be plate haptics which are capable of multiple flexations without damage, and formed, for example, of silicone. The optic 10 and haptics 16 preferably are uniplanar, and two or more haptics 16 extend distally from opposite sides of the optic 10. The outer ends of the haptics 16 may include flexible fingers 17 such as disclosed in U.S. Pat. No. 6,387,126 to Cumming. Preferably the edge 24 of the optic is a 360° square edge.

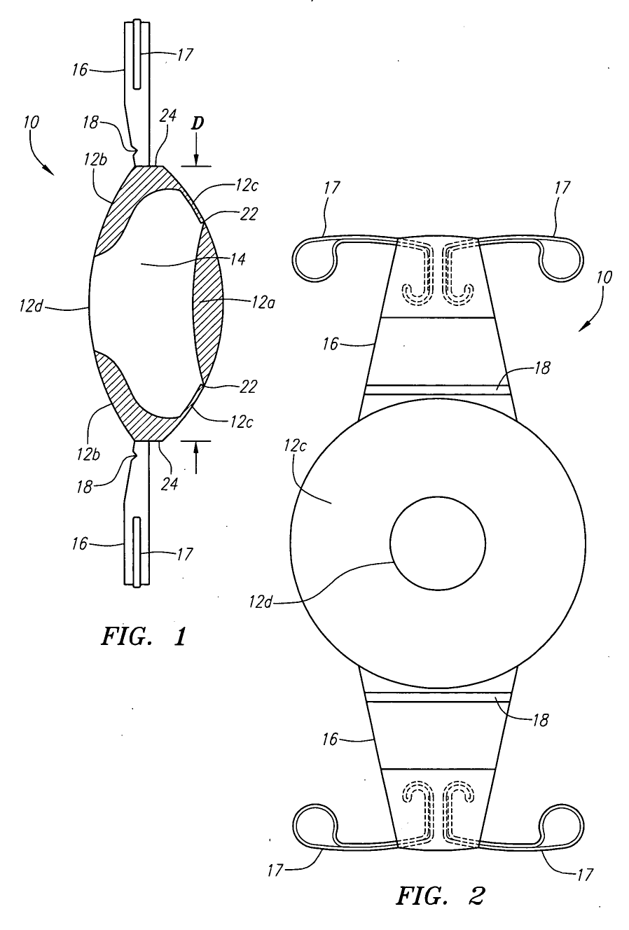

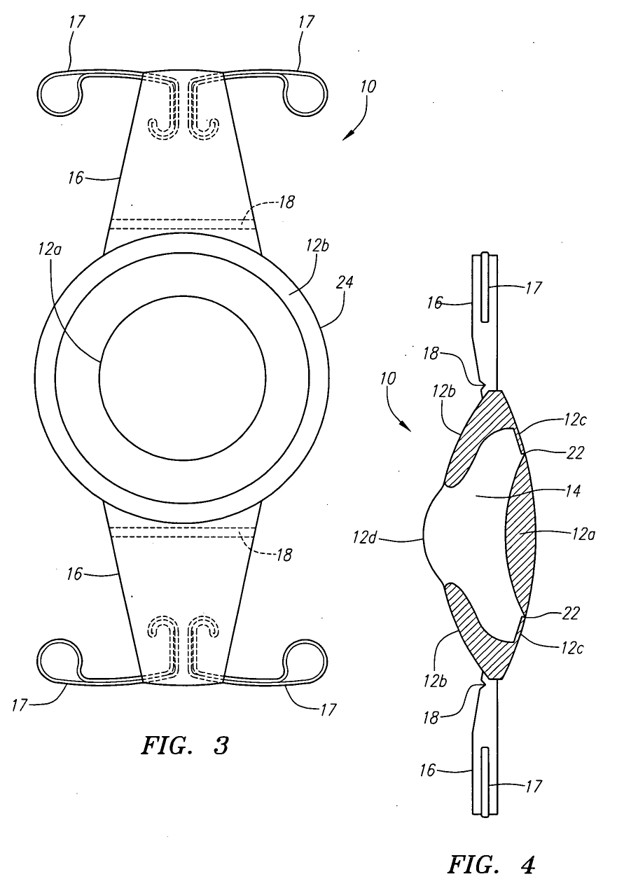

The lens 10 includes portions 12a, 12b and 12d of solid silicone and wherein the portion 12c is substantially thinner, and 12d is even thinner than 12c, to enable a degree of flexibility as can be seen in comparing FIG. 1 and FIG. 4. The interior 14 is of liquid silicone. As is known, the specific gravity of the silicone used in this lens can be the same as or very similar to that of the acqueous solution in the human eye. This results in either no or negligible defomation of the liquid portion of the lens by gravity. The liquid silicone 14 has the same or similar refractive index as the solid components 12. The solid posterior radius of portion 12a prevents defomation of the posterior refracting surface. The radius of the portions 12a or 12b can be changed, during manufacturing, to select the desired power for the lens. Also, the power can be changed, during manufacturing, by changing the volume of the liquid silicone 14 in the lens optic 10.

In accommodating, the posterior surface portion 12a is pushed forward (to the left in FIGS. 1 and 4) by vitreous cavity pressure with constriction of the ciliary muscle. The anterior portion 12d bulges with increased curvature, that is decreased radius, of the anterior portion 12d such as illustrated in FIG. 4.

Example dimensions are 4.5-10.5 mm in overall diameter of portion 12b from D to D in FIG. 1, up to a 5 mm diameter portion 12d, and a 3-6 mm thickness (from right to left) in FIG. 1. A typical thickness for the solid silicon portions 12a and 12b is between 0.5 mm and 1.5 mm. The thickness of the anterior membrane 12d is very thin, preferably about that of a toy balloon, and the thickness of the annulus 12c is approximately two times that thickness to give sufficient flexibility to the lens. The thickness at the hinge 18 area can be 0.1 mm. The hinge area 18 can be a “V” shape as shown but can be a square groove. Also, hinges 22 preferably are provided between 12c and 12a to facilitate anterior movement of the posterior optic 12a.

Furthermore, the power of the present lens can be changed after implantation in the eye by either injecting or removing liquid silicone from the optic 10.

The diameter of the portion 12d as well as its area can be less or more than that of the posterior portion 12a, dependent on the refractive range desired in the design of the lens.

As is well known in the art, an intraocular lens is implanted in the capsular bag of the eye after removal of the natural lens. The lens is inserted into the capsular bag by a generally circular opening cut in the anterior capsular bag of the human lens and through a small opening in the cornea or sclera. The outer ends of the haptics, or loops, are positioned in the cul-de-sac of the capsular bag. The outer ends of the haptics, or the loops, are in close proximity with the bag cul-de-sac, and in the case of any form of loops, the loops are deflected. Knobs can be provided on the outer end portions of the loops for improved securement in the capsular bag or cul-de-sac by engagement with fibrosis, which develops in the capsular bag following the surgical removal of the central portion of the anterior capsular bag.

As noted above, the haptics 16 may have a space or thin area 18 forming a hinge across their surface adjacent to the optic. This facilitates movement of the optic anteriorly and posteriorly relative to the outer ends of the haptics.

Accordingly, there has been shown and described a lens that comprises an optic of solid and liquid silicone and haptic plates, preferably with fixation fingers at the ends of each haptic.

Various changes, modifications, variations, and other uses and applications of the subject invention will become apparent to those skilled in the art after considering this specification together with the accompanying drawings and claims. All such changes, modifications, variations, and other uses of the applications which do not depart from the spirit and scope of the invention are intended to be covered by the claims which follow.

1. An accommodating intraocular lens having an optic formed of solid silicone and liquid silicone,
the optic being circular and having a solid peripheral annular portion extending to anterior and posterior surfaces of the optic, the optic having a central anterior portion and a central posterior portion, the central anterior portion comprising a very thin membrane and the central posterior portion comprising a solid central optical portion surrounded by an annular membrane thicker than the anterior membrane, the optic being designed so that the thin anterior portion can change in radius of curvature upon an increase in vitreous cavity pressure on the posterior solid optic portion.

2. The lens as in claim 1 further including extending portions from an edge of the optic for facilitating placement of the lens in an eye.

3. The lens as in claim 2 wherein the extending portions are haptics.

4. The lens as in claim 2 wherein the extending portions are plate haptics.

5. The lens as in claim 2 whereby the optic can move anteriorly and posteriorly relative to the outer ends of the extending portions.

6. The lens as in claim 1 wherein the solid silicone and the liquid silicone have approximately the same specific gravity as the aqueous solution of a human eye.

7. The lens as in claim 1 designed such that compression of the posterior portion by vitreous pressure can cause a bulging of an anterior surface of the central anterior portion.

8. The lens as in claim 1 wherein the solid and liquid portions of the optic have substantially the same index of refraction.

9. The lens as in claim 1 wherein the optic has radii of curvature to give a patient within whom the lens is implanted emmetropia upon relaxation of the ciliary muscle.

10. The lens as in claim 1 wherein the posterior and anterior portions each have a radius of curvature that can be changed during manufacturing to provide emmetropia for a designated eye.

11. The lens as in claim 1 wherein the power of the optic can be changed after implantation by changing the amount of liquid silicone in the optic.

12. The lens as in claim 1 wherein the thin central anterior portion has a smaller area than the central posterior portion.

13. The lens as in claim 1 wherein the thin central anterior portion has a larger area than the central posterior portion.

14. The lens as in claim 1 wherein the thin central anterior thinner portion has a smaller diameter than the central posterior portion.

15. A lens as in claim 1 wherein the thin central anterior portion is thinner than the annular posterior membrane.

16. A lens as in claim 1 wherein the thin central anterior portion is larger than the annular posterior membrane.

17. A lens as in claim 3 wherein the haptics have a hinge adjacent to the optic.

18. A lens as in claim 1 where there is a 360 degree square edge on the posterior surface of the optic.

19. A lens as in claim 1 wherein the posterior portion has a fixed central radius and the radius of the central anterior portion is deformable.

20. A lens as in claim 1 wherein a solid peripheral area continues to join at a hinged junction with the solid posterior portion of the optic.

21. A lens as in claim 1 wherein an increase in vitreous cavity pressure can cause the lens optic to tilt to facilitate accommodation.

22. A combination accommodating intraocular lens having an optic and formed of both solid and liquid silicone with thin single silicone membranes on both a posterior portion and an anterior portion of the lens, the liquid silicone being contained between the posterior and anterior portions, the membrane on the anterior portion is centered and the posterior membrane is annular and peripheral to the optical center of the posterior optic, the anterior membrane is thinner than the posterior membrane and capable of bulging to change power upon compression of the posterior surface by vitreous pressure.

23. A lens as in claim 22 wherein the lens includes extending portions from the edge of the optic for facilitating placement of the lens in an eye.

24. A lens as in claim 23 wherein the extending portions are haptics.

25. A lens as in claim 24 wherein the haptics have a hinge adjacent to the optic.

26. A lens as in claim 22 wherein the posterior membrane extends between a posterior axial portion and posterior and anterior solid outer portions of the optic, and the thin anterior portion is centered on an anterior side of the lens.

27. An accommodating intraocular lens in which the optic comprises:
an integral central optical system supported by a solid fixed radius, flexible annular peripheral optical component,
a central anterior optical component comprising a thin deformable membrane,
a central liquid optical material within the lens,
a posterior central solid flexible optic suspended from a solid annular support by an annular membrane which is thicker and less resilient than the thin anterior membrane,
the posterior optic being designed to move anteriorly and compress the liquid central optical material to cause the thin anterior membrane to bulge forward to decrease its radius of curvature and increase its refractive power upon an increase of vitreous cavity pressure upon ciliary muscle contraction, and
haptics to support the optical system within the eye.

28. A lens as in claim 27 including fixation devices on the distal ends of the haptics.
